(19) United States
(12) Patent Application Publication
(10) Pub. No.: US 2018/0221004 A1
(43) Pub. Date: Aug. 9, 2018

(54) Flexible Biopsy Device

(71) Applicant: Duke University, Durham, NC (US)

(72) Inventors: Kamran Mahmood, Durham, NC (US); Paolo Maccarini, Durham, NC (US); Don V. Pearce, Durham, NC (US)

(21) Appl. No.: 15/892,171

(22) Filed: Feb. 8, 2018

Related U.S. Application Data
(60) Provisional application No. 62/456,264, filed on Feb. 8, 2017.

(51) Int. Cl. A61B 10/04; A61B 10/02; A61B 1/018; A61B 1/267; A61B 8/12

(52) U.S. Cl. CPC A61B 10/04; A61B 10/0275; A61B 1/018; A61M 2025/0095; A61B 8/12; A61B 2010/045; A61B 1/2676

(57) ABSTRACT

A device for taking a core biopsy sample comprising a flexible catheter assembly and a needle is provided. The flexible catheter assembly comprises a first flexible catheter and a second flexible catheter partially received within the first catheter, and the first flexible catheter and second flexible catheter are coupled to a spring.

### CROSS-REFERENCES TO RELATED APPLICATIONS

The present application claims priority to U.S. Provisional Patent Application No. 62/456,264, filed Feb. 8, 2017, entitled “Flexible Biopsy Device,” which is hereby incorporated by reference in its entirety.

### STATEMENT REGARDING FEDERALLY SPONSORED RESEARCH

Not Applicable.

### BACKGROUND OF THE INVENTION

Despite significant advances in medicine, lung cancer remains the leading cause of cancer-related deaths for both men and women in the U.S. Currently, the 5-year survival rate of lung cancer is under 20%.

To combat this high mortality rate, specialists have tried various types of precision and personalized medicine with limited success. Targeted therapy has proven useful for some patients with certain cancer-driving genetic mutations, such as epidermal growth factor receptor (EGFR), anaplastic lymphoma kinase (ALK), and c-ros oncogene 1 (ROS1) fusion mutations. It has also been shown that cancerous tumors can express programmed death-ligand 1 (PD-L1). Cancer can evade the body's immunity when PD-L1 binds with programmed cell death 1 (PD-1) receptors expressed on T-cells. This PD-L1 can bind with programmed cell death 1 receptors expressed on T-cells, which leads to downregulation of T-cells and allows cancer to evade the body's immunologic defense. Personalized immune checkpoint inhibitors (anti-PD1 and anti-PDL1) have been developed which can disrupt this immunologic tumor evasion. These drugs have been shown to have superior patient outcomes compared to conventional chemotherapy.

To assess a tumor for various mutations and PD-L1 expression, a tissue sample is required. Currently, tissue samples are generally taken using fine-needle aspirates. These needles are frequently used to perform bronchoscopies, and are commonly used in conjunction with an endobronchial ultrasound bronchoscope (EBUS). While these needles are able to obtain some cellular material, they often provide scant tumor cells and do not inform a medical professional about the tumor tissue architecture. With a limited cell sample, it is difficult to obtain information about the genetic and immunologic profile of the tumor or diagnose certain tumors like lymphomas and necrotic malignancies. To overcome the cellular deficiency of samples taken with fine-needle aspirates, several biopsy passes are required, which increases the likelihood of patient complications, procedure time, anesthesia time, and overall cost. Even with multiple biopsy passes, the aspiration needle may still fail to provide sufficient tumor histological architecture. This leaves the patient with few options other than incurring the high cost, delay in treatment, and higher likelihood of complications associated with more invasive surgical biopsy procedures.

Accordingly, a need exists for a device and method of obtaining a core biopsy sample that can be more readily assessed for cancer-driving genetic mutations. In particular, a need exists for a device and method of obtaining sufficient cellular material such that cellular mutations and immunologic markers within a tumor can be assessed.

### SUMMARY OF THE INVENTION

A device and method for taking a core biopsy sample are disclosed herein. The device comprises a flexible catheter assembly and a needle. The flexible catheter assembly comprises a first flexible catheter having an interior surface and an exterior surface, as well as a second flexible catheter having an interior surface and an exterior surface. The second flexible catheter is at least partially received within the first flexible catheter interior surface, and the first flexible catheter and second flexible catheter are coupled to a spring. A position of the first flexible catheter is adjustable relative to a position of the second catheter by altering a tension of the spring. The needle is coupled to a distal end of the second flexible catheter.

In some aspects, the first flexible catheter has a distal end having a tapered section configured as a cutting sheath, which has a leading edge and a trailing edge. The tapered section may have an elliptical conic section and could have a substantially planar surface. The leading edge and trailing edge may be contained within a plane that forms an angle between about 5 degrees and about 85 degrees with respect to a plane normal to the longitudinal axis of the first catheter. Preferably, the leading edge and the trailing edge are contained within a plane that forms an angle between about 15 degrees and about 60 degrees with respect to a plane normal to the longitudinal axis of the first catheter. The flexible catheter assembly may comprise wound stainless steel, and the cutting sheath may comprise silver solder. The flexible catheter assembly may further comprise a plastic sleeve enclosing at least a portion of the first flexible catheter exterior surface. The first flexible catheter may have a tubular shape with a longitudinal axis. The first flexible catheter may have an outer diameter of between about 0.7 mm and about 1.5 mm, and may be between about 0.9 mm and about 1.10 mm. The second flexible catheter may also have a tubular shape having a longitudinal axis. The second flexible catheter may also have an outer diameter of between about 0.4 mm and about 1.2 mm, or an outer diameter between about 0.8 mm and about 1.0 mm.

In some aspects, the needle comprises a tissue trap. The tissue trap has a first angled section, a second angled section, and an exposed needle section. The exposed needle section is disposed between the first angled section and the second angled section. The exposed needle section may be a planar surface. The tissue trap may have a semi-cylindrical shape with angled bases. In some aspects, the semi-cylindrical shape of the tissue trap has a radius approximately equal to a radius of the needle, which may be between about 0.25 mm and about 0.75 mm. Preferably, the needle has a radius between about 0.4 mm and about 0.5 mm. The first angled section of the needle may form a first acute angle with respect to the exposed needle section and the second angled section of the needle may form a second acute angle with respect to the exposed needle section. The first acute angle formed between the first angled section of the needle and the exposed needle section may be between about 5 degrees and about 85 degrees, and could be between about 20 degrees and about 70 degrees. The second acute angle formed between the second angled section of the needle and the exposed needle section may be between about 5 degrees and about 85 degrees, and may be between about 20 degrees and about 70 degrees. In some aspects, the first acute angle formed between the first angled section and the exposed needle section and the second angle formed between the second angled section and the exposed needle section are equivalent. The first angled surface may comprise a fillet. At least one of the first angled section and the second angled section may comprise a curved surface. The needle may comprise a medical grade stainless steel, and could have an exposed needle section with an axial length of between about 5 mm and about 50 mm. The needle may have a distal end comprising a tapered surface, and the tapered surface may have a leading edge and a trailing edge, where the leading edge is configured to pierce body tissue. The needle may be coupled to the distal end of the second catheter with silver solder.

In some aspects, the device for taking a core biopsy further comprises a handle for housing the spring and at least a portion of the first catheter and second catheter and the handle is sized to be held within a human hand. The handle may comprise a controller in communication with the spring and can be configured to adjust a position of the first flexible catheter relative to the second flexible catheter. The handle may further comprise a second controller, where the second controller is in communication with the second flexible catheter and the second controller is configured to adjust a position of the second flexible catheter relative to the handle. The spring coupled to the first flexible catheter and the second flexible catheter may be a component of a longitudinally retracting spring-loaded mechanism, and the longitudinally retracting spring-loaded mechanism may be housed within the handle.

A method of taking a core biopsy sample using a device for taking a core biopsy sample is also disclosed. The device comprises a flexible catheter assembly and a needle. The flexible catheter assembly has a first flexible catheter and a second flexible catheter at least partially received within the first flexible catheter, and the needle is coupled to a distal end of the second flexible catheter and has a tissue trap. The method comprises piercing tissue with a distal end of the first flexible catheter, adjusting an axial position of the first flexible catheter relative to an axial position of the second catheter by withdrawing a portion of the first flexible catheter, which causes a portion of the needle to protrude from the distal end of the first flexible catheter to contact the tissue sample. The method further comprises adjusting the axial position of the first flexible catheter relative to the axial position of the second flexible catheter, such that the needle no longer protrudes from the distal end of the first flexible catheter, thereby capturing a portion of the tissue sample.

In some aspects, the device for taking a core biopsy sample has a combination of features previously described. In some aspects, the first flexible catheter has a distal end having a tapered section configured as a cutting sheath, which has a leading edge and a trailing edge. The tapered section may have an elliptical conic section and could have a substantially planar surface. The leading edge and trailing edge may be contained within a plane that forms an angle between about 5 degrees and about 85 degrees with respect to a plane normal to the longitudinal axis of the first catheter. Preferably, the leading edge and the trailing edge are contained within a plane that forms an angle between about 15 degrees and about 60 degrees with respect to a plane normal to the longitudinal axis of the first catheter. The flexible catheter assembly may comprise wound stainless steel, and the cutting sheath may comprise silver solder. The flexible catheter assembly may further comprise a plastic sleeve enclosing at least a portion of the first flexible catheter exterior surface. The first flexible catheter may have a tubular shape with a longitudinal axis. The first flexible catheter may have an outer diameter of between about 0.7 mm and about 1.5 mm, and may be between about 0.9 mm and about 1.10 mm. The second flexible catheter may also have a tubular shape having a longitudinal axis. The second flexible catheter may also have an outer diameter of between about 0.4 mm and about 1.2 mm, or an outer diameter between about 0.8 mm and about 1.0 mm.

In some aspects, the needle has a number of additional features. The tissue trap may have a first angled section, a second angled section, and an exposed needle section. The exposed needle section may be disposed between the first angled section and the second angled section. The exposed needle section may be a planar surface. The tissue trap may have a semi-cylindrical shape with angled bases. In some aspects, the semi-cylindrical shape of the tissue trap has a radius approximately equal to a radius of the needle, which may be between about 0.25 mm and about 0.75 mm. Preferably, the needle has a radius between about 0.4 mm and about 0.5 mm. The first angled section of the needle may form a first acute angle with respect to the exposed needle section and the second angled section of the needle may form a second acute angle with respect to the exposed needle section. The first acute angle formed between the first angled section of the needle and the exposed needle section may be between about 5 degrees and about 85 degrees, and could be between about 20 degrees and about 70 degrees. The second acute angle formed between the second angled section of the needle and the exposed needle section may be between about 5 degrees and about 85 degrees, and may be between about 20 degrees and about 70 degrees. In some aspects, the first acute angle formed between the first angled section and the exposed needle section and the second angle formed between the second angled section and the exposed needle section are equivalent. The first angled surface may comprise a fillet. At least one of the first angled section and the second angled section may comprise a curved surface. The needle may comprise a medical grade stainless steel, and could have an exposed needle section with an axial length of between about 5 mm and about 50 mm. The needle may have a distal end comprising a tapered surface, and the tapered surface may have a leading edge and a trailing edge, where the leading edge is configured to pierce body tissue. The needle may be coupled to the distal end of the second catheter with silver solder.

In some aspects, the device used in the method for taking a core biopsy further comprises a handle for housing the spring and at least a portion of the first catheter and second catheter and the handle is sized to be held within a human hand. The handle may comprise a controller in communication with the spring and can be configured to adjust a position of the first flexible catheter relative to the second flexible catheter. The handle may further comprise a second controller, where the second controller is in communication with the second flexible catheter and the second controller is configured to adjust a position of the second flexible catheter relative to the handle. The spring coupled to the first flexible catheter and the second flexible catheter may be a component of a longitudinally retracting spring-loaded mechanism, and the longitudinally retracting spring-loaded mechanism may be housed within the handle. The position of the first flexible catheter may be adjustable relative to a position of the second catheter by altering a tension of the spring.

In some aspects, the step of adjusting an axial position of the first flexible catheter relative to an axial position of the second catheter by withdrawing a portion of the first flexible catheter is performed by prompting a controller in communication with the spring, and prompting the controller causes the tension of the spring to be altered. The handle may further comprise a deployment control, where the deployment control is in communication with the spring and is configured to alter a tension of the spring when prompted. The second step of adjusting the axial position of the first flexible catheter may be performed by prompting the deployment control, where the deployment control causes the tension of the spring to be altered a second time. In some aspects, the method further comprises the step of removing the device and the captured tissue biopsy from a respiratory system. The method may also comprise the step of locating a tissue target using an endobronchial ultrasound (EBUS) scope.

In another aspect, a method of manufacturing a device for taking a core biopsy sample is disclosed. The method comprises providing a first flexible catheter having an interior surface and an exterior surface and providing a second flexible catheter having an exterior surface smaller than the interior surface of the first flexible catheter. The method further comprises attaching a needle to a distal end of the second flexible catheter, providing a sheath to a distal end of the first catheter, and coupling the first flexible catheter and the second flexible catheter to a longitudinally retracting spring-loaded mechanism, where the second flexible catheter is at least partially received within the interior surface of the first flexible catheter.

In some aspects of the method, the first flexible catheter has a distal end having a tapered section configured as a cutting sheath, which has a leading edge and a trailing edge. The tapered section may have an elliptical conic section and could have a substantially planar surface. The leading edge and trailing edge may be contained within a plane that forms an angle between about 5 degrees and about 85 degrees with respect to a plane normal to the longitudinal axis of the first catheter. Preferably, the leading edge and the trailing edge are contained within a plane that forms an angle between about 15 degrees and about 60 degrees with respect to a plane normal to the longitudinal axis of the first catheter. The flexible catheter assembly may comprise wound stainless steel, and the cutting sheath may comprise silver solder. The method may further comprise providing a plastic sleeve enclosing at least a portion of the first flexible catheter exterior surface. The first flexible catheter may have a tubular shape with a longitudinal axis. The first flexible catheter may have an outer diameter of between about 0.7 mm and about 1.5 mm, and may be between about 0.9 mm and about 1.10 mm. The second flexible catheter may also have a tubular shape having a longitudinal axis. The second flexible catheter may also have an outer diameter of between about 0.4 mm and about 1.2 mm, or an outer diameter between about 0.8 mm and about 1.0 mm.

In some aspects, the step of providing a cutting sheath to the distal end of the first flexible catheter includes soldering the distal end of the first flexible catheter with a high temperature silver. The process may further include grinding the distal end of the first flexible catheter to an angle. In some aspects of the method, the step of attaching the needle is performed by soldering the needle to the distal end of the second flexible catheter using high temperature silver.

In some aspects, the needle is machined to have a tissue trap. The tissue trap has a first angled section, a second angled section, and an exposed needle section. The exposed needle section is disposed between the first angled section and the second angled section. The exposed needle section may be a planar surface. The tissue trap may have a semi-cylindrical shape with angled bases. In some aspects, the semi-cylindrical shape of the tissue trap has a radius approximately equal to a radius of the needle, which may be between about 0.25 mm and about 0.75 mm. Preferably, the needle has a radius between about 0.4 mm and about 0.5 mm. The first angled section of the needle may form a first acute angle with respect to the exposed needle section and the second angled section of the needle may form a second acute angle with respect to the exposed needle section. The first acute angle formed between the first angled section of the needle and the exposed needle section may be between about 5 degrees and about 85 degrees, and could be between about 20 degrees and about 70 degrees. The second acute angle formed between the second angled section of the needle and the exposed needle section may be between about 5 degrees and about 85 degrees, and may be between about 20 degrees and about 70 degrees. In some aspects, the first acute angle formed between the first angled section and the exposed needle section and the second angle formed between the second angled section and the exposed needle section are equivalent. The first angled surface may comprise a fillet. At least one of the first angled section and the second angled section may comprise a curved surface. The needle may comprise a medical grade stainless steel, and could have an exposed needle section with an axial length of between about 5 mm and about 50 mm. The needle may have a distal end comprising a tapered surface, and the tapered surface may have a leading edge and a trailing edge, where the leading edge is configured to pierce body tissue. The needle may be coupled to the distal end of the second catheter with silver solder.

In some aspects, the method for taking a core biopsy further comprises the step of molding a handle, where the handle has a partially hollow housing cavity and is sized to be held within a human hand. The method may further comprise securing the longitudinally retracting spring-loaded mechanism within the handle housing cavity, where at least a portion of the first catheter and the second catheter are then contained within the handle. The handle may comprise a controller in communication with the spring and can be configured to adjust a position of the first flexible catheter relative to the second flexible catheter. The handle may further comprise a second controller, where the second controller is in communication with the second flexible catheter and the second controller is configured to adjust a position of the second flexible catheter relative to the handle.

### BRIEF DESCRIPTION OF THE DRAWINGS

FIG. 1A is a pictorial view of a prior art endobronchial ultrasound bronchoscope.

FIG. 1B is a detail view of a distal end of the prior art endobronchial ultrasound bronchoscope of FIG. 1A.

FIG. 2 is a perspective view of an exemplary biopsy device of the present disclosure.

FIG. 3A is a cross-sectional view of flexible catheters that can be incorporated into the biopsy device of FIG. 2, taken along section line 3A-3A in FIG. 2.

FIG. 3B is a cross-sectional view of alternative flexible catheters that can be incorporated into the biopsy device of FIG. 2, taken along section line 3B-3B in FIG. 2.

FIG. 4 is a perspective view of a needle design that can be used in the biopsy device of FIG. 2.

FIG. 5A is a plan view of a distal end of the biopsy device of FIG. 2, employing a needle design similar to that shown in FIG. 4.

FIG. 5B is a plan view of the distal end of the biopsy device of FIG. 2, where the first catheter is in a withdrawn position.

FIG. 6A is a perspective view of a handle design that can be used in the biopsy device of FIG. 2.

FIG. 6B is a cross-sectional view of the handle of FIG. 6A, taken along lines 6B-6B.

FIG. 7 is a process diagram detailing a method of taking a core biopsy sample using a device such as the biopsy device of FIG. 2.

FIG. 8 is a process diagram detailing a method of manufacturing a device for taking a core biopsy sample, such as the biopsy device of FIG. 2.

FIG. 9A is a comparison of tissue samples on slides taken using the process and apparatus of FIG. 7 and a prior art process and apparatus.

FIG. 9B is a microscopic view of the top tissue sample in FIG. 9A, taken using the process and apparatus of FIG. 7.

FIG. 9C is a microscopic view of the tissue sample taken using the prior art process and apparatus as shown in the bottom sample in FIG. 9A.

Like reference numerals will be used to refer to like parts from Figure to Figure in the following description of the drawings.

### DETAILED DESCRIPTION OF THE INVENTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention. While the concepts of the disclosure are described in relation to a needle adapted for use in an endobronchial ultrasound bronchoscope, it should be understood that it is equally applicable to other biopsy devices and mechanisms and can be used in combination with such devices.

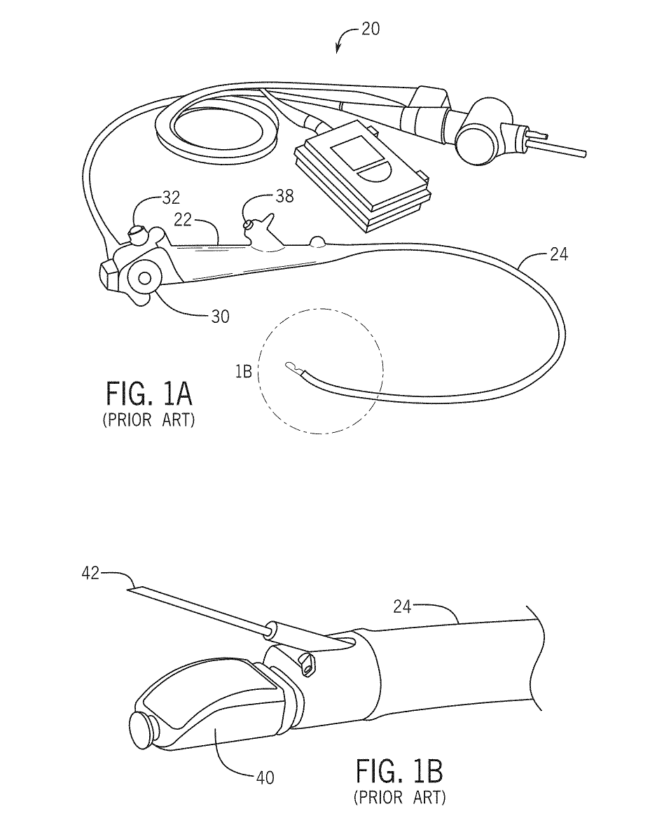

Turning now to FIGS. 1A and 1B, a prior art endobronchial ultrasound (EBUS) scope 20 is shown. The EBUS scope 20 has a handle 22 and a flexible insertion tube 24 which incorporates an instrument channel 38 that can be used to insert various biopsy devices. To operate the EBUS scope 20, a physician can insert the insertion tube 24 into a patient's mouth, where it is then passed through the trachea and into the bronchi. As shown in FIGS. 1A and 1B, an ultrasound transducer is coupled to the distal end of the insertion tube 24 to provide real-time ultrasound guidance, allowing a physician to avoid neighboring vascular structures while simultaneously locating the biopsy target.

In the illustrative EBUS scope 20, the insertion tube 24 is formed of flexible polymeric material, such as polypropylene. By using a flexible material, the insertion tube 24 can be maneuvered through much of the respiratory system without contacting tissue walls or exerting inadvertent stress on areas of tissue. The insertion tube 24 movement can be controlled by an angulation control lever 30 that controls up-down angulation. Using the angulation control lever 30, the distal end of the insertion tube 24 can be directed through the respiratory passage to a desired tissue location.

The EBUS bronchoscope 20 is a video bronchoscope that uses both video and fiber-optic technologies. A charge-coupled device (CCD) chip (not shown) is located behind an objective lens located at the distal end of the bronchoscope. The lens projects the image of the airway onto the CCD chip, which converts the image into electric signals. These signals are carried via wires that travel through the insertion tube 24 and a connector at the proximal end of the scope to a separate video processor. The airway is illuminated by an external light source. Light passes through the connector at the proximal end of the bronchoscope, via glass fiber bundles to the distal end of the scope 20. The tip of the illustrative bronchoscope 20 has a 7.5 MHz convex ultrasound transducer 40. The ultrasound images are transmitted through proximal connectors to an ultrasound processor, and visualized along with the conventional bronchoscopy images.

When a desired tissue sample is located by a user operating the EBUS scope 20, a biopsy device can be inserted into the instrument channel 38. The biopsy device or needle 42 will extend beyond the distal end of the instrument channel 38 at the tip of the bronchoscope and extend into the desired target, where it can obtain a sample. A suction 32 is provided on the EBUS scope to remove any secretions or blood from the airways.

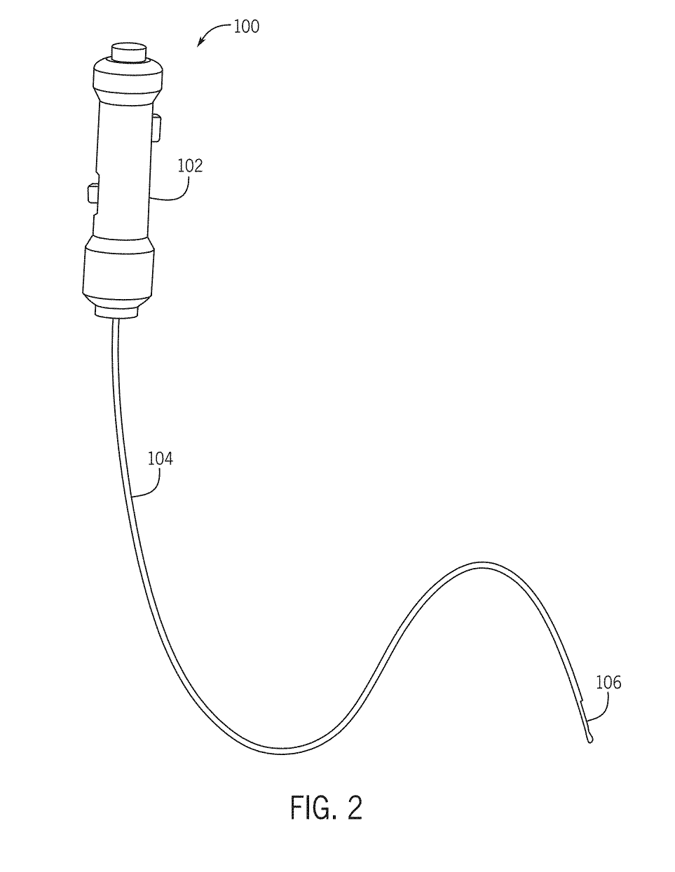

Turning now to FIG. 2, a biopsy device 100 for taking a core biopsy sample in accordance with embodiments of the disclosure is provided. The biopsy device 100 comprises a handle 102, a flexible catheter assembly 104, and a needle 106. The needle 106 is coupled to the flexible catheter assembly 104, and the flexible catheter assembly 104 is coupled to handle 102. In some aspects, the flexible catheter assembly 104 is coupled to the handle 102 by a spring loaded mechanism contained within the interior of the handle 102. While the needle 106 is shown exposed from the flexible catheter assembly 104 in the figure, it should be appreciated that the needle can also be at least partially or fully received within the flexible catheter assembly 104 during the use of the biopsy device 100.

In some aspects, the biopsy device 100 can be coupled to an EBUS scope, such as the EBUS scope 20 described in FIG. 1A. The biopsy device 100 may be used in combination with the EBUS scope 20, with the needle 106 and flexible catheter assembly 104 received within the instrument channel 38. In some aspects, the bottom of the handle 102 comprises a locking mechanism to securely couple the biopsy device 100 to the EBUS scope 20.

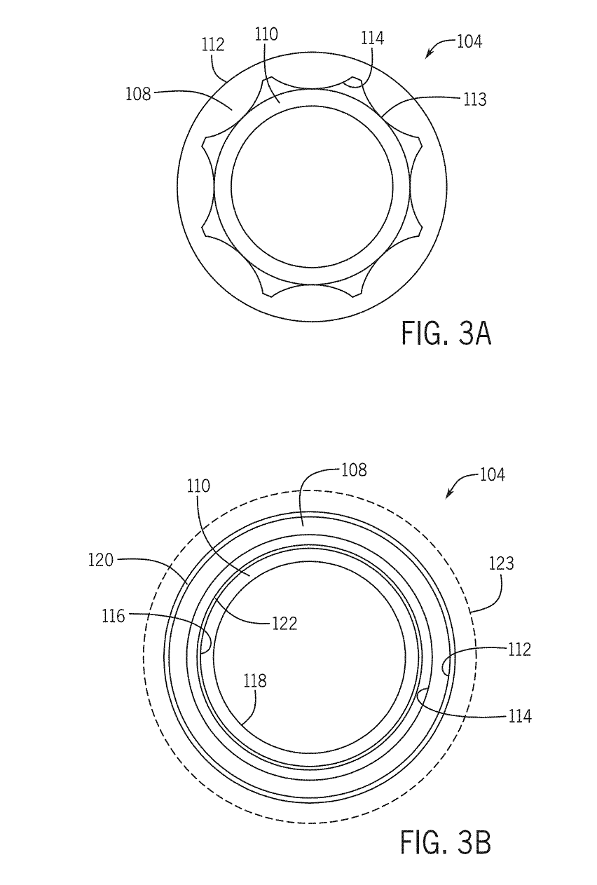

Turning now to FIGS. 3A and 3B, cross-sectional views of the flexible catheter assembly 104 are provided. The flexible catheter assembly 104 includes two flexible catheters 108 and 110. The first flexible catheter 108 has an exterior surface 112 and an interior surface 114. The second flexible catheter 110 has an exterior surface 116 and an interior surface 118 and is at least partially received within the interior surface 114 of the first flexible catheter 108, as shown. The first flexible catheter 108 may be partially tubular, and the exterior surface 112 of the first flexible catheter can be a curved surface. As shown in FIGS. 3A and 3B, the interior surface 114 can be uniformly curved or may take on other shapes. In some aspects, the interior surface 114 includes bumps 113 that can reduce friction between the first flexible catheter 108 and the second flexible catheter 110. The interior surface 114 shown in FIG. 3A incorporates bumps 113, so that sliding friction may be reduced between the interior surface 114 of the first flexible catheter 108 and the second flexible catheter 110 by limiting the contact area between the catheters 108, 110.

Alternatively, the flexible catheter assembly 104 can include a first flexible catheter 108 and a second flexible catheter 110 having tubular cross-sections where each catheter has a longitudinal axis, as shown in FIG. 3B. In such aspects, the first flexible catheter 108 has an exterior surface 112 and an interior surface 114 defined by a first flexible catheter inner diameter and first flexible catheter outer diameter. Once again, the second flexible catheter 110 is at least partially received within the inner surface 114 of the first catheter, as the second catheter outer surface 116 is sized to be smaller than the first catheter interior surface 114. This allows the two catheters 108, 110 to be telescoping. In some aspects, the first flexible catheter 108 and the second flexible catheter 110 may be positioned concentric with one another, such that the catheters 108, 110 share a common longitudinal axis.

In some aspects, a friction-reducing coating 120 and 122, such as PTFE, may be applied to surfaces of the first flexible catheter 108 and the second flexible catheter 110. In the illustrative aspect, a PTFE spray coating has been applied to the first catheter exterior surface 112 and the second catheter exterior surface 116. The coating can have a number of different dimensions, depending on the sizing of the catheters 108, 110 being used in the flexible catheter assembly 104. In aspects containing a friction-reducing coating, the coating 120, 122 could have a thickness ranging between about 0.005 mm to about 0.5 mm or greater, and could be uniform or applied sporadically about the desired surfaces 112, 116. In some examples, a coating of 0.03 mm thickness is used. The coating may allow improved movement of the first flexible catheter 108 relative to the instrument channel 38 of the EBUS scope 20, as well as improved movement of the first flexible catheter 108 relative to the second flexible catheter 110, as described below. Additionally, while the friction-reducing coating 120 and 122 has been described as a coating, it should be appreciated that lubricants such as surgical jellies can also be used to coat surfaces of the flexible catheters 108, 110 to decrease frictional forces between the catheters 108, 110, and are also within the scope of the present disclosure.

The flexible catheter assembly 104 may be comprised of several different flexible materials. In some aspects, both the first flexible catheter 108 and second flexible catheter 110 are comprised of wound stainless steel. Wound stainless steel catheters, such as those disclosed in U.S. Pat. No. 6,881,194 B2 by Asahi Intecc Co. Ltd., which are hereby incorporated by reference, provide the flexible catheters 108, 110 with the ability to articulate without linking or flat-spotting. The catheters 108, 110 can be flexible and kink-free and can be capable of transmitting torque and axial loading along the length of the material that is superior to many plastics being used in this application currently. In some aspects, ACTONE® flexible stainless steel tubing (produced by Asahi Intecc USA, Inc. of Santa Ana, Calif.) can be used, such as the ACTONE FLAT or ACTONE SWG configurations. As shown in FIG. 3A, the first flexible catheter 108 may comprise a combination of ACTONE FLAT technology and ACTONE SWG technology, while the second flexible catheter 110 may comprise ACTONE FLAT technology. In addition to the examples shown, it should be appreciated that many other stainless steel catheter configurations are possible, such as the ACTONE UT configuration or other configurations for producing stainless steel catheters with the desired properties discussed above. Additionally, it should be appreciated that several materials may be used for the flexible catheter assembly 104, including 304 stainless steel, 316 stainless steel, and other medical grade metallic and polymeric materials that have torque transmission capabilities.

In some aspects, the flexible catheter assembly 104 is sized to fit within the instrument channel 38 of an EBUS scope, similar to EBUS scope 20 of FIG. 1. Although the dimensions may vary slightly, the instrument channel 38 of EBUS scope 20 is about 2.2 mm in diameter. Accordingly, the diameter of the flexible catheter assembly 104 can be smaller than the diameter of the instrument channel 38. In some aspects of the present disclosure, an 18-gauge design is incorporated. In such aspects, the first flexible catheter 108 has an outer diameter of about 1.02 mm and the second flexible catheter 110 has an outer diameter of about 0.91 mm. The first flexible catheter inner diameter is chosen to be less than the second flexible catheter outer diameter, such that there is enough clearance for the first flexible catheter 108 to move relative to the second flexible catheter 110 during the cutting operation, as discussed below with reference to FIGS. 5A and 5B. In some aspects, the first flexible catheter 108 and the second flexible catheter 110 are concentric with one another, and share a common longitudinal axis. In other aspects, larger or smaller gauge designs can be used. In some aspects, 16-gauge designs can be incorporated where the first flexible catheter 108 has an outer diameter exceeding 1.50 mm. Similarly, smaller designs such as a 22-gauge design can be incorporated where the first flexible catheter 108 has an outer diameter of about 0.7 mm. More preferably, the first flexible catheter 108 has an outer diameter between about 0.9 mm and about 1.10 mm. The second flexible catheter 110 can have an outer diameter ranging between about 0.4 mm and about 1.2 mm, and preferably between about 0.8 mm and about 1.0 mm.

In some aspects, the flexible catheter assembly 104 further comprises a plastic sleeve 123 positioned around a portion of the first flexible catheter exterior surface 112. The plastic sleeve 123 may prevent damage to the interior of the instrument channel 38 as the flexible catheter assembly 104 is passed through the channel. The plastic sleeve 123 can have a diameter of anywhere between 0.8 mm (for smaller gauge designs) to 2.1 mm (for larger gauge designs), or may be omitted entirely. In the 18-gauge design, the plastic sleeve 123 can have an outer diameter between about 1.5 mm and about 1.8 mm. The diameter of the plastic sleeve 123 should be chosen so that the flexible catheter assembly 104 can pass through the instrument channel 38 of the EBUS scope 20 without exerting axial force that causes the flexible insertion tube 24 to move or change shape significantly. It should be appreciated that a number of different flexible polymeric materials can be used to provide this feature.

Like many biopsy devices used today, the flexible catheter assembly 104 may have a length between about 30 cm and about 60 cm, depending on the type of bronchoscope intended to be used to perform the procedure. This length allows the flexible catheter assembly 104 to extend through the instrument channel 38 of the EBUS scope 20 and out the distal end of the instrument channel, so that the flexible catheter assembly 104 may biopsy the targeted lesion. In some aspects, the first flexible catheter 108 can be slightly longer than the second flexible catheter 110. However, it should be appreciated that both catheters can have approximately the same length or the second flexible catheter 110 can have a length greater than the length of the first flexible catheter 108 and still remain within the scope of the present disclosure.

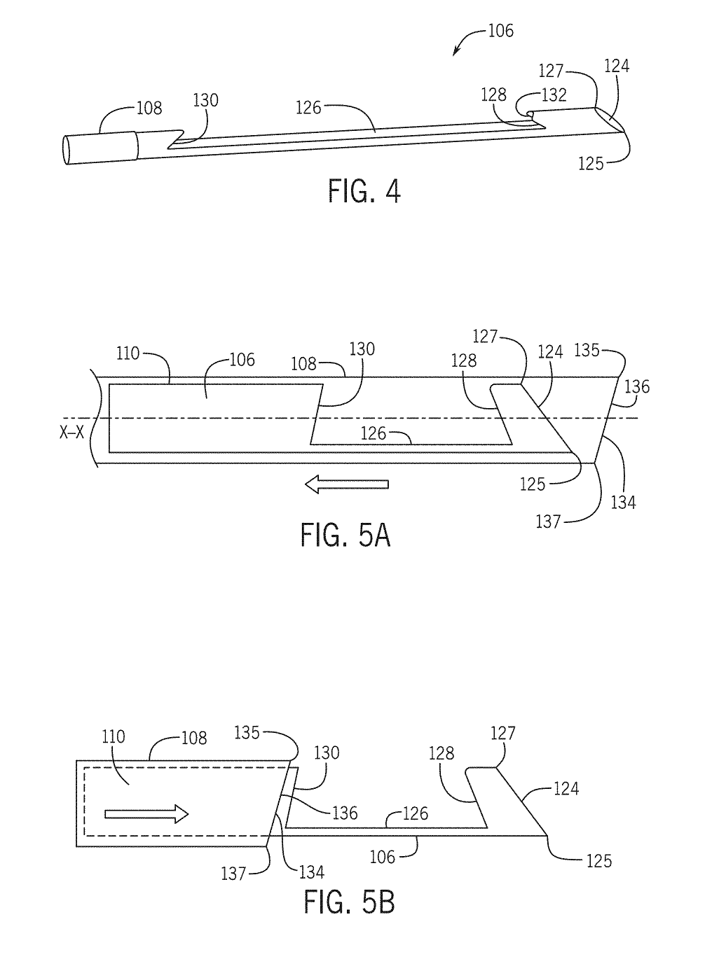

Turning now to FIG. 4, a needle 106 is shown exposed from the first flexible catheter 108. Unlike traditional needle designs used for fine-needle aspirates, this needle 106 is designed and adapted to take core biopsy samples. To perform such a task, the needle 106 has multiple cutting features. For example, the needle may comprise a tissue trap formed from an exposed needle section 126, a first angled section 128, and a second angled section 130, where the exposed needle section 126 is disposed between the first angled section 128 and the second angled section 130.

In some aspects, the exposed needle section 126 comprises a planar surface. The tissue trap can be defined by a hollow half cylinder shape with angled bases 128, 130. In some aspects, the semi-cylindrical shape of the tissue trap has a radius approximately equal to a radius of the needle 106. The radius may be between about 0.25 mm and about 0.75 mm in some aspects, and may preferably be between about 0.4 mm and about 0.5 mm. Such a shape can provide stiffness in the needle and can help promote tissue capture.

The first angled section 128 and the second angled section 130 can provide sharpened edges which may act as barbs to prevent tissue movement. When a user desires to take a tissue sample, the needle 106 will be introduced into the targeted tissue. As the needle 106 moves axially through the tissue target, the needle 106 may cause tissue to deform. Once the needle passes through the tissue sample, the tissue's elastic properties urge the tissue to return to its former shape. The tissue will expand toward the exposed needle section 126 while contained between the first angled section 128 and the second angled section 130. Because the angled sections 128, 130 provide sharpened edges, the tissue may be partially pierced, and unable to return to its original form. This traps the tissue between the exposed needle section 126 and angled sections 128, 130, so that the trapped tissue can be removed from the body with additional cutting steps.

In some aspects, the needle 106 has a tapered surface 124, comprising a leading edge 125 and a trailing edge 127. The leading edge 125 can be configured to pierce body tissue as the needle 106 comes into contact with tissue. The tapered surface 124 can be a solid surface or can contain one or more through holes (not shown), which could be placed into communication with suction features or the like to aid in the tissue removal process. In some aspects, the tapered surface 124 is a planar surface, providing the distal end of the needle 106 with an elliptical conic section. In other aspects, the tapered surface may be curved, such that the leading edge 125 arcs gradually toward the trailing edge 127. Additionally, stress relieving features can be added to one or both of the angled sections 128, 130, such as fillets 132. This can reduce the chance of a needle shattering during use. Additionally, the needle 106 can be made of echogenic material, such as medical grade stainless steel with dimples to be used in concert with an EBUS scope, such as that disclosed in FIG. 1.

In certain aspects, the needle 106 is designed to have an outer diameter similar to that of the second flexible catheter 110. This allows the needle 106 to be at least partially received within the first flexible catheter during use of the biopsy device 100. In the illustrative aspects, the needle 106 is coupled to the second flexible catheter 110. The coupling process may occur in a number of ways, including high temperature silver soldering, as explained in further detail with respect to FIG. 8.

The needle 106 may come in a number of different sizes. For example, the needle 106 may be between about 1 cm and about 6 cm long. More preferably, the needle 106 is between about 2 cm and about 4 cm long. The needle diameter may fall between the range of sizes discussed with regard to the catheters 108, 110 above. In the 18-gauge design, the needle 106 has a diameter of about 0.91 mm, like the second flexible catheter 110. The exposed needle section 126 may have an axial length chosen from the range of between about 5 and about 50 mm. In certain aspects, the exposed needle section 126 has an axial length of about 25 mm.

In some aspects, the first angled section 128 of the needle 106 forms a first acute angle with respect to the exposed needle section 126. Similarly, the second angled section 130 of the needle 106 may form a second acute angle with respect to the exposed needle section 126. In some aspects, the first acute angle is between about 5 degrees and about 85 degrees, and may preferably be between about 20 degrees and about 70 degrees. The second acute angle may similarly be between about 5 degrees and about 85 degrees, and may preferably be between about 20 degrees and about 70 degrees. In some aspects, the first acute angle and the second acute angle differ by less than 5 degrees, and are approximately equivalent. While the angled sections 128, 130 are shown in the figure as forming acute angles with respect to the exposed needle section 126, it should be appreciated that these angled sections 128, 130 may form right angles or obtuse angles with the exposed needle section 126 and still be considered within the scope of the present disclosure. Similarly, while the angled sections 128, 130 are shown as planar surfaces, one or more of the angled sections 128, 130 could have a curved surface.

Turning now to FIGS. 5A and 5B, the cutting functionality of the biopsy device 100 is shown. When a flexible catheter assembly 104 is first passed through the instrument channel 38 of an EBUS scope 20 as shown in FIG. 1, the second flexible catheter 110 and the needle 106 are received entirely within the first flexible catheter 108. This protects both the bronchoscope 20 and the needle 106 as the flexible catheter assembly 104 is passed through the instrument channel 38. Such an arrangement will prevent the needle 106 from contacting a surface of the instrument channel 38 that could cause the needle 106 to stick into the surface and even shatter, if enough axial loading is provided. Once the flexible catheter assembly 104 reaches the distal end of the instrument channel 38, it can then extend outward to contact a tissue target.

To take a tissue sample, a distal end of the first flexible catheter 108 can be provided with a cutting sheath 134. The cutting sheath may be arranged as a tapered surface 136 in a traditional needle shape, as shown, or could be otherwise ground to provide an edge capable of piercing through tissue. In aspects having a tapered surface 136, the cutting sheath 134 comprises a leading edge 135 and a trailing edge 137. In some aspects, this tapered surface is a substantially planar surface, providing the distal end of the first flexible catheter 108 with an elliptical conic shape. The leading edge 135 and trailing edge 137 may be contained within a plane that forms an angle between about 5 degrees and about 85 degrees with respect to a plane normal to the longitudinal axis X-X of the first catheter 108. In some aspects, the leading edge 135 and trailing edge 137 are contained within a plane that forms an angle between about 15 degrees and about 60 degrees with respect to a plane normal to the longitudinal axis X-X of the first catheter 108. In some aspects, the cutting sheath 134 further comprises a silver solder component. When the first flexible catheter 108 comprises wound stainless steel, the silver solder may be added to fortify the cutting sheath 134. Prior to shaping the cutting sheath 134 with a tapered surface 136 or other cutting shape, silver solder may be applied to the distal end of the first catheter 108 to strengthen the bond between the wound stainless steel wires that make up the first catheter 108.

Once the cutting sheath 134 passes through the distal end of the instrument channel 38, the cutting sheath 134 contacts tissue. Because the flexible catheter assembly 104 is comprised of materials capable of transmitting axial loads, a user can continue to urge the flexible catheter assembly 104 out of the instrument channel 38, so that tissue is pierced by the cutting sheath 134. The flexible catheter assembly 104 can be urged forward until a desired depth into the tissue has been reached.

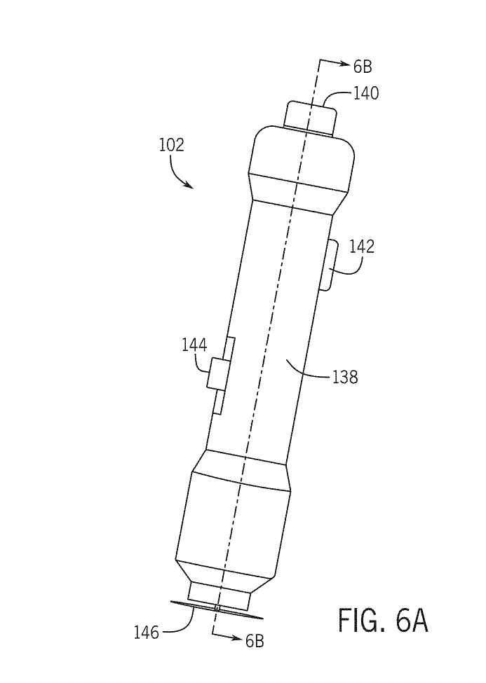

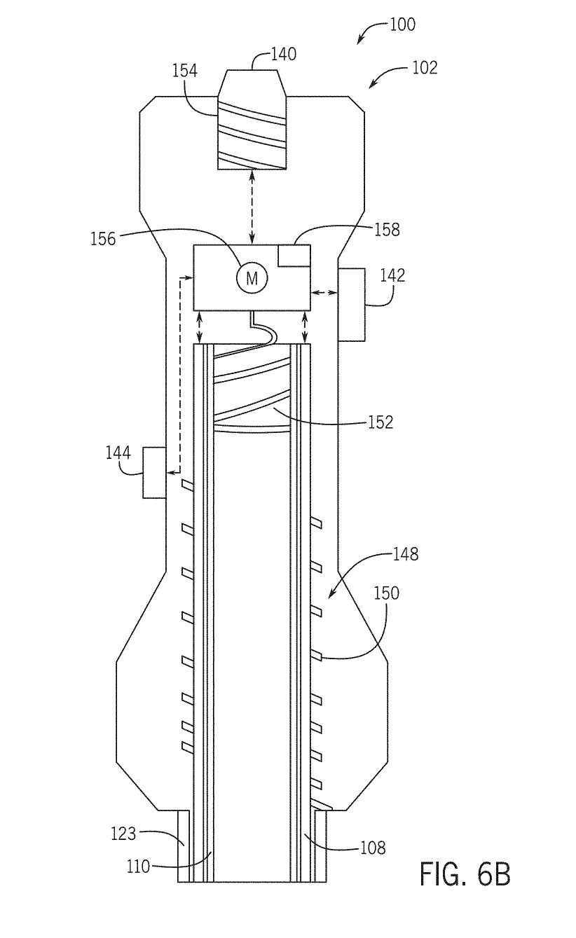

Once the desired tissue depth has been reached, the needle 106 is exposed, as shown in FIG. 5B. This can be performed in a number of ways. In the illustrative aspect, the first flexible catheter 108 and second flexible catheter 110 are coupled to a spring-loaded mechanism 148 shown in FIG. 6B. The spring-loaded mechanism 148 can be received within the handle 102 and can be coupled to the flexible catheter assembly 104. The spring-loaded mechanism 148 can include multiple springs 150, 152, 154 that can be selectively tensioned independently or in combination, using one or more buttons or controls described in detail with reference to FIG. 6A. In one aspect, each catheter 108, 110 is coupled to a separate spring 150, 152, which can be concentrically positioned within the handle 102.

Using the spring-loaded mechanism 148, the biopsy device can locate and obtain a core biopsy sample effectively. When the EBUS scope 20 and biopsy device 100 are being maneuvered through the trachea and into the bronchi toward the desired tissue sample, the spring-loaded mechanism 148 can be loaded (e.g., tensioned) so that both of the first flexible catheter 108 and the second flexible catheter 110 are urged further into the handle 102. In the retracted position, the tapered surfaces 136, 124 of the first flexible catheter 108 and the second catheter 110 can be received within the plastic sleeve 123 (as shown in FIG. 3B), which may protect the EBUS scope 20, the instrument channel 38 that receives the biopsy device 100, and the patient. The insertion tube 24 of the EBUS scope 20 can be directed toward the tissue removal site.

Once the insertion tube 24 (and therefore, the biopsy device 100) is positioned near the tissue removal site, the spring-loaded mechanism 148 can be released, which causes the tapered surfaces 136, 124 of both the first flexible catheter 108 and the second flexible catheter 110 to translate rapidly outward from the plastic sleeve 123 into the tissue to be removed.

When a desired tissue sample depth has been reached, a user can prompt a controller 158 to load the spring 150. This controller would be in communication with the spring-loaded mechanism 148, and would alter the tension of the spring 150, causing the first flexible catheter 108 to be partially withdrawn from the tissue sample, while leaving the second flexible catheter 110 and needle 106 locked in position at the desired tissue sample depth. In some examples, the spring-loaded mechanism 148 can be configured to immediately retract (e.g., reload the spring coupled to) the first flexible catheter 108 into the plastic sleeve 123 after the first flexible catheter 108 and second flexible catheter 110 are translated into the tissue sample to be removed. In some embodiments, the first flexible catheter 108 is withdrawn by between about 5 mm and about 60 mm, and preferably between about 15 mm and about 45 mm. The needle 106 then becomes at least partially exposed to a tissue sample, because the needle 106 at least partially protrudes from the distal end of the first flexible catheter 108 when the spring-loaded mechanism 148 is loaded. The needle design then provides elastic relief to the tissue sample, which elastically expands into the tissue trap defined by the exposed needle section 126 and angled sections 128, 130. The edges of the angled sections 128, 130 may then temporarily hold the tissue in place. Although the figure shows the exposed needle section 126 entirely exposed from the first flexible catheter 108, it should be appreciated that multiple spring settings can be provided, such that the exposed needle section 126 may be only partially exposed from the first flexible catheter 108. In some aspects, the spring-loaded mechanism 148 is a component of a longitudinally retracting spring-loaded mechanism.

Once the tissue sample has expanded into the tissue trap, the spring-loaded mechanism can be released, again by altering the tension of at least one spring 150 coupled to the first flexible catheter assembly 108. This can be performed by a user prompting a controller 158 to release the spring-loaded mechanism 148. When the spring-loaded mechanism 148 is released, the spring 150 imparts an axial force on the first flexible catheter 108. This force causes the first flexible catheter 108 to rapidly return to its original axial position shown in FIG. 5A, enclosing the needle 106 and second flexible catheter 110. Due to its sharpness and its rapid movement through the tissue, the cutting sheath 134 slices much of the tissue contained within the tissue trap, and isolates it from the remaining tissue. This tissue sample is then trapped between the exposed needle section 126, angled sections 128, 130, and first flexible catheter 108. The first flexible catheter 108 and the second flexible catheter 110 (including the needle 106 and the tissue sample) can then be retracted into the plastic sleeve 123 using one or more controls or buttons on the handle 102 to load the spring-loaded mechanism once again 148. A series of indents and traces, as well as other components can be present within the handle 102 housing to provide the necessary stoppers and rotation sequences of the springs 150, 152, 154. By removing the biopsy device 100 from the EBUS scope 20, the core biopsy sample can be removed from the body. It should be appreciated that the cutting action described herein where the first flexible catheter 108 moves relative to the second flexible catheter 110 can be reversed such that the second flexible catheter 110 moves relative to the first flexible catheter 108.

In an alternative embodiment, the loading sequence of the spring-loaded mechanism 148 for taking a tissue sample can be adjusted. For example, the first flexible catheter 108 and the second flexible catheter 110 can be initially retracted into the handle 102 and plastic sleeve 123 when the spring-loaded mechanism 148 is loaded and moved toward the tissue sample. Once at the tissue sample, a button or other control on the handle 102 can be actuated to release the spring-loaded mechanism 148 coupled to the second flexible catheter assembly 110, which causes the needle 106 to translate and protrude outward from the plastic sleeve 123 and the first flexible catheter 108, into the tissue sample. A second button or control on the handle 102 can then be actuated, which alters the tension of the spring 150 coupled to the first flexible catheter 108 and translates the first flexible catheter 108 (and cutting sheath 134) outward from the handle 102 and plastic sleeve 123, around the needle 106. The rapid translation of the first flexible catheter 108 slices and traps tissue into the needle 106 and the flexible catheter assembly 104, which can then be removed from the tissue site. Once the tissue has been sliced and contained within the needle 106, the spring-loaded mechanism 148 can be reloaded, retracting the flexible catheters 108, 110 into the handle 102 and plastic sleeve 123, and the biopsy device 100 can be removed from the EBUS scope 20. This type of spring-loaded mechanism 148 can be particularly effective when the needle 106 is formed of strong materials (e.g., titanium, stainless steel) or has a substantially planar or axially-symmetrical shape that can restrict the bending of the needle 106 upon tissue insertion.

Referring now to FIG. 6A, the handle 102 of the biopsy device 100 is shown in further detail. In some aspects, the handle 102 is comprised of a plastic material and can be sized to fit comfortably within a human hand. The handle 102 may comprise a grip 138 that allows for easy handling and transport of the device 100. As stated earlier, the handle 102 may also include a locking device 146 that allows for attachment to the instrument channel 38 of an EBUS scope 20, disclosed in FIG. 1. Such a locking device 146 may be positioned on the bottom of the handle 102, as shown.

The handle 102 may comprise a number of different buttons or controls. As discussed earlier, the handle 102 may house the spring-loaded mechanism 148, as well as a portion of the first flexible catheter 108 and the second flexible catheter 110. In some aspects, the spring-loaded mechanism 148 is a longitudinally retracting spring-loaded mechanism. Additionally, the handle 102 may comprise a sheath retractor control 140. The sheath retractor control 140 can be in electrical or mechanical communication with the spring-loaded mechanism 148 and the first flexible catheter 108. When depressed or prompted by a user, the sheath retractor control 140 can mechanically load the spring-loaded mechanism 148 or indicate to a controller 158 and a motor 156 in communication with the spring-loaded mechanism 148 that the mechanism 148 must be loaded. The loading of the spring-loaded mechanism 148 results in the partial withdrawal of the first flexible catheter 108 and causes at least a portion of the needle 106 to protrude from the distal end of the first flexible catheter 108, as discussed with reference to FIGS. 5A-5B. When the biopsy device 100 is in use, the at least partial exposure of the needle 106 allows a portion of the needle 106 to contact a tissue sample.

Additionally, a sheath deployment control 142 may be provided on the handle 102. The sheath deployment control 142 can be in electrical or mechanical communication with the spring-loaded mechanism 148 and the first flexible catheter 108. When depressed or prompted by a user, the sheath deployment control 142 can mechanically release the spring mechanism or indicate to a controller 158 and a motor 156 in communication with the spring-loaded mechanism 148 that the mechanism 148 must be released. The release of the spring-loaded mechanism 148 results in the tissue slicing process, where the first flexible catheter 108 is rapidly forced through a tissue sample to cut the tissue sample into the tissue trap of the needle 106. In some aspects, the second flexible catheter 110 and needle 106 remain stationary during this process.

In some non-limiting examples, the handle 102 may further comprise a length adjustment control 144. The length adjustment control 144 may be placed in communication with the second flexible catheter 110, and can adjust the length of the second flexible catheter 110 relative to the first flexible catheter 108. Similarly, it may adjust the positioning of the second flexible catheter 110 relative to the handle 102 by increasing or decreasing the amount of the second flexible catheter 110 contained within the interior of the handle 102. This length adjustment control 144 allows a user to adjust the length of the exposed needle when the sheath retractor control 140 is prompted. In some aspects, the length adjustment control 144 may provide multiple length settings. For example, a length adjustment control 144 may include four steps, which could adjust an exposed needle section 126 length between 17.5 mm and 25 mm in 2.5 mm increments. Other adjustment units and lengths can similarly be used and are fully within the scope of the disclosure, including some aspects which include no adjustment control 144 at all. For example, the exposed needle length could be adjusted via a knob and its position could be provided on a digital display on the handle or otherwise, similar to an electronic caliper. Similarly, the second flexible catheter 110 may be rigidly locked in place in other aspects.

In some aspects, one or more indicators are also attached to the device to indicate whether the spring-loaded mechanism 148 is in a loaded or unloaded state. Such an indicator may be an LED light positioned on the side of the handle, a positional change of the sheath retractor control 140 that is visible to a user (i.e. the button has a loaded and unloaded position, similar to a ballpoint pen), or otherwise. Such indications may prevent a physician from failing to obtain a sample, or from failing to initiate the tissue cutting process, which could prove useful and may help avoid any additional patient discomfort.

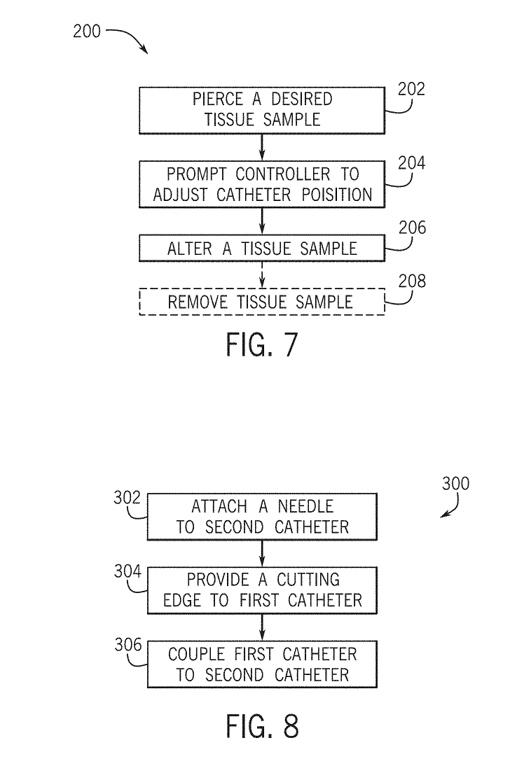

Turning now to FIG. 7, a method of taking a core biopsy sample 200 is shown. The method of taking a core biopsy sample 200 is performed using a biopsy device 100 having any combination of the features discussed with reference to FIGS. 2-6. First, a desired tissue sample can be located. This can be performed using an EBUS scope 10, such as that described in FIG. 1. Using endoscopic visualization and an ultrasonic transducer on the tip of the scope, a physician can readily maneuver the insertion tube of the scope through a patient's respiratory system and locate a desired tissue target. This tissue target will be accessed with the EBUS bronchoscope 10 in a patient's bronchi.

Next, a biopsy device 100 is inserted into the instrument channel 38 of the bronchoscope 10. The biopsy device 100 comprises a handle 102, a flexible catheter assembly 104 having a first flexible catheter 108 and a second flexible catheter 110 coupled to the handle 102, and a needle 106 coupled to the second flexible catheter 110. The biopsy device 100 may contain any number of the features disclosed with reference to FIGS. 2-6. The flexible catheter assembly 104 and needle 106 are introduced into the instrument channel 38 of the bronchoscope 10 and out the distal end of the instrument channel 38 at the tip of the bronchoscope 10.

The flexible catheter assembly 104 then contacts the desired tissue target. In some aspects, the flexible catheter assembly 104 will be contained within a plastic sheath when the desired tissue sample is first contacted. A user provides an axial force to advance the flexible catheter assembly 104 out of the plastic sheath into the target, or the spring-loaded mechanism 148 can force the flexible catheter assembly 104 into the target. A cutting sheath 134 on the distal end of the flexible catheter assembly allows the flexible catheter assembly 104 to pierce the tissue at block 202, and move through the tissue sample. The user can also provide axial force that allows the flexible catheter assembly 104 to reach a target depth in the tissue.

Once a satisfactory depth has been reached in the tissue sample, a controller 158 can be prompted to adjust the axial position of a first flexible catheter 108 in the flexible catheter assembly 104 at block 204. This controller 158 may be a button or other control on the device handle 102, as described above. When prompted, the controller 158 can electronically or mechanically alter the tension of a spring-loaded mechanism 148 present inside the biopsy device handle 102. Loading this device causes the first flexible catheter 108 to be withdrawn from the tissue sample, adjusting the axial position of the first flexible catheter 108 relative to the axial position of the second flexible catheter 110. In some embodiments, the adjustment of the axial position of the first flexible catheter 108 is between about 5 mm and about 60 mm, and can be more preferably between about 15 mm to about 45 mm. This adjustment causes at least part of the needle 106 to become exposed to the tissue sample, where the needle 106 can contact the tissue sample. As stated with reference to FIG. 4, the needle 106 of the biopsy device 100 may comprise a tissue trap, which can capture tissue as it attempts to expand into a recess in the needle 106.

Once tissue has expanded into the tissue trap in the needle 106, the tissue sample can be altered at block 206. In some aspects, the tissue trap provides sharp edges, so that the needle 106 may shear a tissue sample as it is removed from the patient. In other aspects, the spring-loaded mechanism 148 may be released by again altering the spring 150 tension, which could be performed by prompting a deployment control. Releasing the spring-loaded mechanism 148 causes the first flexible catheter 108 and cutting sheath 134 to rapidly return to its original axial position relative to the axial position of the second flexible catheter 110 and handle 102, slicing tissue and capturing a tissue sample within the needle recess. When the spring-loaded mechanism 148 is released, the portion of the needle 106 previously protruding from the distal end of the first flexible catheter 104 is once again received within the first flexible catheter 104. In some aspects, a core biopsy can be obtained. The flexible catheter assembly 104, needle 106, and core biopsy sample may then be removed from the airway or other location at block 208, and the sample can be tested and used for improved diagnostic and genetic analysis.

It should be appreciated that this method 200 can be performed using the biopsy device 100 described with reference to FIGS. 2-6, and is tailored to be performed with such an instrument. While different features and dimensions can be varied, added, or omitted to the biopsy device 100, it should be appreciated that each combination of the features disclosed above has been contemplated for use in the present method 200, and should be understood as included within the description of the method 200.

Referring now to FIG. 8, a method of manufacturing the device 100 for taking core biopsy samples 300 described with reference to FIGS. 2-6 is provided. The process 300 includes providing two flexible catheters 108, 110 of differing diameters. In some aspects, these catheters 108, 110 are made of wound stainless steel. The catheters 108, 110 may be between about 30 cm and 60 cm long, although the first catheter 108 may be longer than the second, smaller diameter catheter 110. At block 302, a needle 106 is attached at the distal end of the second catheter 110, which can be done via high temperature silver soldering, brazing, or other methods of establishing a connection. The needle shape, such as that disclosed with reference to FIG. 4, may be produced by milling or other molding or machining processes.

Using a similar high temperature coupling technique such as silver soldering, a cutting edge can be provided to the distal end of the first catheter 108 at block 304. For example, the distal end of the first catheter 108 may be soldered to bond and encapsulate strands of stainless steel wire, rendering a solidified tip. After the solidified tip has cooled, it can be ground to a traditional needle shape, and provided with a cutting edge at block 304. The cutting edge may be shaped to have the dimensions of the cutting sheath 134 described with reference to FIGS. 5A-5B, with a leading edge 135 and a trailing edge 137.

Finally, the second catheter 110 can be placed within the first catheter 108 at block 306. In some aspects, the second catheter 110 is then coupled to a longitudinally retracting spring-loaded mechanism 148. In some aspects, the longitudinally retracting spring-loaded mechanism 148 can be housed within a polymeric handle 102. The handle 102 can be formed by injection molding, blow molding, or otherwise, and can be formed to have a cavity capable of housing the longitudinally retracting spring-loaded mechanism, as well as a portion of the catheter assembly 104. The spring-loaded mechanism can be placed within the handle 102, and can be connected to various electronic or mechanical controllers, as discussed above, which alter the axial positions of the catheters 108, 110 with respect to one another and with respect to the handle 102.

Once again, it should be appreciated that this method of manufacturing a device for taking a core biopsy sample 300 has been created to produce the biopsy device 100 described with reference to the preceding disclosure. Accordingly, the method of manufacturing a device 300 can be used to form such a device having any combination of the features disclosed above with reference to FIGS. 2-6. This method contemplates the addition, omission, or alteration of dimensions as well, and should be considered to encompass the production of any of the devices 100 described with reference to the preceding figures.

### EXAMPLE

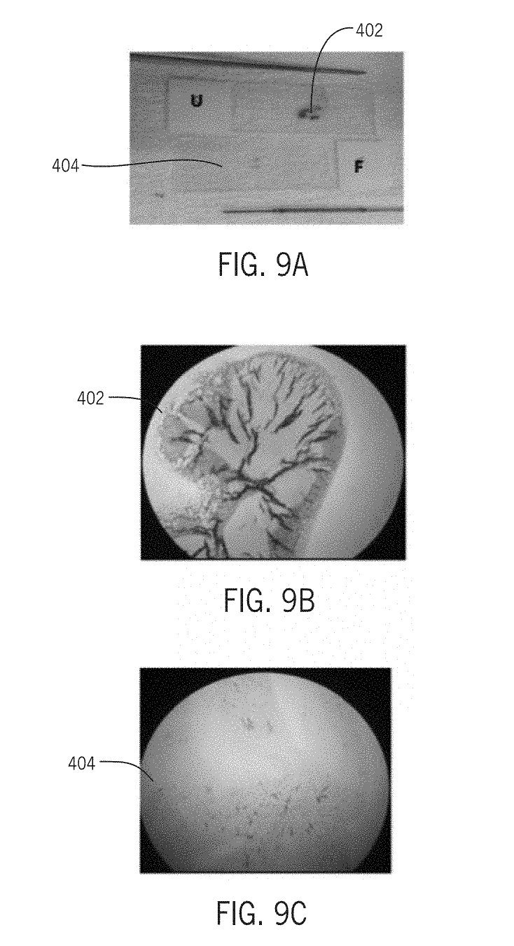

Turning now to FIGS. 9A-9C, a comparison of tissue samples taken from a chicken liver is shown. Using a biopsy device 100 as described above with reference to FIGS. 2-6 and using an aspect of the method of use 200 disclosed in FIG. 7, a tissue sample 402 was obtained. Using a prior art 22 gauge needle design from Olympus (available commercially as ViziShot EBUS TBNA Needle, a second tissue sample 404 was obtained. Five biopsies were taken using each apparatus and method, and the results were inspected using a microscope (not shown).

Using the disclosed method 200 and apparatus 100, core biopsy tissue samples were obtained in each attempt. As can be seen in the microscopic image shown in FIG. 9B, the obtained tissue sample 402 contained strands of tissue having sufficient histologic architecture and cellular material to enable advanced analysis and testing for cancer. As can be seen in FIG. 9C, the sample 404 taken using the Olympus 22 gauge needle contained scant cellular material, and did not constitute a core biopsy sample. The tissue sample 404 taken using the prior art method and apparatus failed to provide sufficient cellular structure and material to enable desirable analytical techniques that could be performed on the samples 402 obtained using the apparatus and methods of the present disclosure.

The core tissue sample 402 taken using the disclosed apparatus 100 and methods 200 consistently produced superior tissue samples when compared to the prior art apparatus and methods. Returning to FIG. 9A, the difference in sufficiency of the tissue samples is readily apparent. The core tissue sample 402 obtained using the disclosed apparatus 100 and methods 200 was visible to the naked eye, whereas the sample 404 obtained by the Olympus biopsy device showed only a watery aspirate with vaguely visible coloration. Accordingly, the present methods 200 and apparatuses 100 have been shown to provide adequate samples containing undisturbed cellular core biopsy.

Thus, the disclosure provides devices and processes for taking core biopsy samples, as well as a method of manufacturing the same. Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A device for taking a core biopsy sample, the device comprising:
a flexible catheter assembly, the flexible catheter assembly comprising:
a first flexible catheter, the first flexible catheter having an interior surface and an exterior surface;
a second flexible catheter, the second flexible catheter having an interior surface and an exterior surface and being at least partially received within the first flexible catheter interior surface, wherein the first flexible catheter and second flexible catheter are coupled to a spring and a position of the first flexible catheter is adjustable relative to a position of the second catheter by altering a tension of the spring; and
a needle, the needle being coupled to a distal end of the second flexible catheter.

2. The device of claim 1, wherein the first flexible catheter has a distal end having a tapered section configured as a cutting sheath, wherein the cutting sheath comprises a leading edge and a trailing edge.

3. The device of claim 2, wherein the leading edge and the trailing edge are contained within a plane that forms an angle between about 5 degrees and about 85 degrees with respect to a plane normal to a longitudinal axis of the first catheter.

4. The device of claim 3, wherein the leading edge and the trailing edge are contained within a plane that forms an angle between about 15 degrees and about 60 degrees with respect to a plane normal to the longitudinal axis of the first catheter.

5. The device of claim 1, wherein the needle comprises a tissue trap having a first angled section, a second angled section, and an exposed needle section, the exposed needle section disposed between the first angled section and the second angled section.

6. The device of claim 5, wherein the exposed needle section is a planar surface.

7. The device of claim 5, wherein the tissue trap has a semi-cylindrical shape with angled bases.

8. The device of claim 7, wherein the semi-cylindrical shape of the tissue trap has a radius approximately equal to a radius of the needle.

9. The device of claim 5, wherein the first angled section of the needle forms a first acute angle with respect to the exposed needle section and the second angled section of the needle each forms a second acute angle with respect to the exposed needle section.

10. The device of claim 5, wherein the first angled section comprises a fillet.

11. The device of claim 5, wherein the exposed needle section has an axial length of between about 5 mm and about 50 mm.

12. The device of claim 1, further comprising a handle for housing the spring and at least a portion of the first catheter and second catheter and wherein the handle is sized to be held within a human hand.

13. The device of claim 12, wherein the handle comprises a controller in communication with the spring and is configured to adjust a position of the first flexible catheter relative to the second flexible catheter.

14. The device of claim 13, wherein the handle further comprises a second controller, the second controller being in communication with the second flexible catheter and wherein the second controller is configured to adjust a position of the second flexible catheter relative to the handle.

15. The device of claim 12, wherein the spring coupled to the first flexible catheter and the second flexible catheter is a component of a longitudinally retracting spring-loaded mechanism, and the longitudinally retracting spring-loaded mechanism is housed within the handle.

16. The device of any of claim 1, wherein the flexible catheter assembly further comprises a plastic sleeve enclosing at least a portion of the exterior surface of the first flexible catheter.

17. The device of claim 1, wherein an outer diameter of the first flexible catheter is between about 0.9 mm and about 1.10 mm.

18. The device of claim 17, wherein an outer diameter of the second flexible catheter is between about 0.8 mm and about 1.0 mm.

19. A device for taking a core biopsy sample, the device comprising:
a first flexible catheter partially received within and extending away from a handle, the first flexible catheter being coupled to a biasing element housed within the handle to translate the first flexible catheter between an extended position and a retracted position; and
a second flexible catheter at least partially received within the first flexible catheter, the second flexible catheter having a needle coupled to a distal end of the second catheter;
wherein the needle is exposed from the first flexible catheter further when the first flexible catheter is in the retracted position than when the first flexible catheter is in the extended position.

20. A device for taking a core biopsy sample, the device comprising:
a handle defining a housing, the housing receiving a portion of a first flexible catheter and a portion of a second flexible catheter that is at least partially received within an interior surface of the first flexible catheter; and
a needle coupled to a distal end of the second flexible catheter and selectively received within the interior surface of the first flexible catheter, the needle comprising a tissue trap defined by a first angled section, an exposed section, and a second angled section;
wherein a spring is received within the housing and coupled to the first flexible catheter and the handle, the spring biasing the first flexible catheter away from the housing toward a position where the interior surface of the first flexible catheter receives the needle.
